(19) United States
(12) Patent Application Publication
(10) Pub. No.: US 2024/0226583 A9
(43) Pub. Date: Jul. 11, 2024

(54) Auxiliary Strain Relief Member For The Lead Of An Active Implantable Medical Device

(71) Applicant: Greatbatch Ltd, Clarence, NY (US)

(72) Inventors: Luis Daniel Villamil, Montevideo (UY); Ignacio Agustin Amesto, Montevideo (UY); Camila Duarte, Montevideo (UY)

(21) Appl. No.: 18/382,716

(22) Filed: Oct. 23, 2023

Related U.S. Application Data
(60) Provisional application No. 63/418,066, filed on Oct. 21, 2022.

(51) Int. Cl. A61N 1/375

(52) U.S. Cl. CPC A61N 1/3752

(57) ABSTRACT

An active implantable medical device (AIMD) assembly has a medical device housing containing an electrical power source connected to control circuitry, and a header assembly connected to the device housing. The header assembly supports a plurality of co-axially aligned terminal blocks that are in open communication with a header opening. A conventional strain relief member is connected to the header assembly at the header opening. A number of different auxiliary strain relief devices are described. The auxiliary strain relief devices connect to the conventional strain relief member to help reduce tension and stress on a lead after the AIMD assembly is implanted in a body and the lead electrodes are connected to body tissue.

### CROSS-REFERENCE TO RELATED APPLICATION

This application claims priority to U.S. Provisional Application Ser. No. 63/418,066, filed on Oct. 21, 2022.

### BACKGROUND OF THE INVENTION

### 1. Field of the Invention

The present invention relates to the field of active implantable medical devices (AIMDs). More particularly, the present invention describes various strain relief designs that are supported by a header assembly for an AIMD to help reduce tension and stress on a lead after the medical device is implanted in a body and the lead electrodes are connected to body tissue.

An AIMD has at least one implantable lead that is connected to a series of co-axially aligned terminal blocks in the device header. The proximal portion of the lead connected to the device header has a plurality of proximal electrical contacts that correspond to the number of terminal blocks in the header. The terminal block assembly in the header in turn is connected to control circuitry that is housed inside the device and powered by an electrical power source, such as a primary or rechargeable, secondary electrochemical cell. When the proximal electrical contacts of the lead are inserted into the terminal block assembly in the header, the lead contacts are electrically connected to the control circuitry. The lead is designed to deliver electrical stimulation to body tissue or sense biological signals from the body tissue, or both stimulate and sense.

### 2. Prior Art

It is important for an AIMD to have a strain relief member. Typically, the strain relief member is embedded in the device header where the lead exits the header, or the strain relief member is part of the implantable lead itself. Whether embedded in the device header or a fixed part of the lead, the strain relief member helps to reduce tension and stress on the lead after the medical device is implanted in a body and the distal electrodes are connected to body tissue. The strain relief member also helps to protect the lead from being damaged against sharp edges and to prevent sharp bends.

For example, when an AIMD is implanted in a skull during a craniometry procedure, the strain relief member is particularly important since it protects the lead from the edges of the cranial pocket when the lead transitions from the device header to the outer surface of the skull. If chafing and abrasion of the polymeric outer insulation of a lead against the edges of the cranial pocket is too severe, it could expose the electrical conductors of the lead to body fluids, which could result in an undesirable short circuit condition. A significant drawback with convention designs, however, is that the strain relief member is of a fixed length and geometry that does not permit any flexibility regarding the final implanted configuration.

However, simply lengthening a conventional strain relief member is not necessarily going to solve this problem. A longer strain relief member has the consequence of higher insertion forces when the lead is moved into the header assembly, which in turn can generate buckling issues that possibly prevent the electrical contacts of the lead from properly aligning with the terminal block assembly in the device header.

In that respect, there is a need for various designs for auxiliary strain relief members that are used in conjunction with conventional strain relief members to form a new strain relief assembly. The new strain relief assembly should be long enough to protect the lead from chafing and abrasion of its polymeric outer insulation and to help reduce tension and stress on the lead after the medical device is implanted in a body and the distal electrodes are connected to body tissue. An example of this is when a lead lays against the edges of a cranial pocket in the skull during a craniometry procedure. Additionally, the strain relief assembly must not adversely impact the force needed to insert a lead into the header assembly.

### SUMMARY OF THE INVENTION

The present invention is directed to various auxiliary strain relief members that are connected to a conventional strain relief member to form a new strain relief assembly that improves protection of a lead for an extended length without increasing the force needed to insert the lead into the header assembly. This is particularly important in medical devices that are implanted in the skull or near bones. Moreover, possible lead buckling issues are also avoided.

Consequently, a strain relief system according to the present invention has two main parts, which gives a surgeon the option of using both parts. The first part is the conventional strain relief member that is embedded in the device header or is a fixed part of the lead. The other part is an auxiliary strain relief member that is connected to the conventional strain relief member. That way, the surgeon has the option of using only the embedded strain relief member or both the embedded and auxiliary strain relief members. Using both strain relief members as an assembly increases the length of the lead that is protected from tension and stress and possibly chafing against a sharp edge of the cranial pocket during, for example, a craniometry.

In that regard, the present inventive subject matter is directed to various designs for auxiliary strain relief members that are connected to a conventional strain relief member to provide a strain/bend relief assembly for an implantable lead. The strain relief assembly helps protect the lead from chafing and unintended wear when exiting the medical device and, for example, when passing over the edge of the cavity formed in the skull during a craniectomy. That is without increasing the size of the implantable medical device or without increasing the force needed to insert the lead into the device header. The lead must also be securely fixed and supported inside the strain relief assembly. Moreover, the present auxiliary strain relief members provide options for the strain relief geometry that can be selected by the surgeon during the implant procedure. This means that the strain relief assembly can be customized to suit each patient's anatomy. For example, one of the design concepts permits trimming the length of the auxiliary strain relief member during the implant procedure.

Also, it is important to highlight that the lead is not fixed to the header of the medical device by the present auxiliary strain relief members. Instead, the auxiliary strain relief members only add strain/bend relief to the lead in addition to the protection that is already provided by the conventional strain relief member. That way, the auxiliary strain relief members of the present invention add flexibility to the final implanted system without increasing the force needed to insert the lead into the device header.

These and other aspects of the present invention will become increasingly more apparent to those skilled in the art by reference to the following detailed description and to the appended drawings.

### BRIEF DESCRIPTION OF THE DRAWINGS

FIG. 1 is a perspective view of an active implantable medical device 10 that has been implanted in a cranial pocket 12 formed in the skull 14 of a patient.

FIG. 2 is a plan view of the implanted medical device 10 shown in FIG. 1.

FIG. 2A is a cross-sectional view, partly broken away, of an implantable lead 32 connected to the header assembly 18 of the implantable medical device shown in FIG. 1 with the lead 32 resting against the edge 12A of the cranial pocket 12 in the skull 14.

FIG. 3 is a perspective view of the implantable medical device 10 shown in FIG. 1 with three implantable leads 32 having already been inserted into the header assembly and a fourth lead 32 being moved into the header assembly 18.

FIG. 4 is a cross-sectional view taken along line 4-4 of FIG. 3.

FIG. 5 is a perspective view of an implantable medical device 10 connected to an auxiliary strain relief device 60 according to the present invention.

FIG. 6 is a cross-sectional view taken along line 6-6 of FIG. 5.

FIG. 6A is an enlarged view of the indicated area in FIG. 6.

FIG. 7 is a perspective view of an implantable medical device 10 connected to another embodiment of an auxiliary strain relief device 80 according to the present invention.

FIG. 8 is a perspective view of an implantable medical device 10 connected to another embodiment of an auxiliary strain relief device 100 according to the present invention.

FIG. 9 is a perspective view of an implantable medical device 10 connected to another embodiment of an auxiliary strain relief device 120 according to the present invention.

FIG. 9A is an enlarged view of the indicated area in FIG. 9.

### DETAILED DESCRIPTION OF THE PREFERRED EMBODIMENTS

As used herein, the term “active implantable medical device” means an implantable medical device that is designed to deliver electrical stimulation to the body tissue of a patient or sense biological signals from the body tissue, or both stimulate and sense.

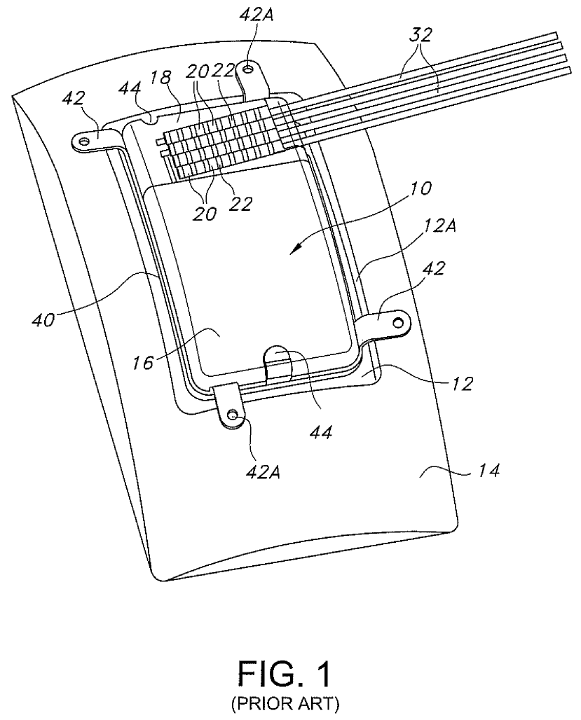

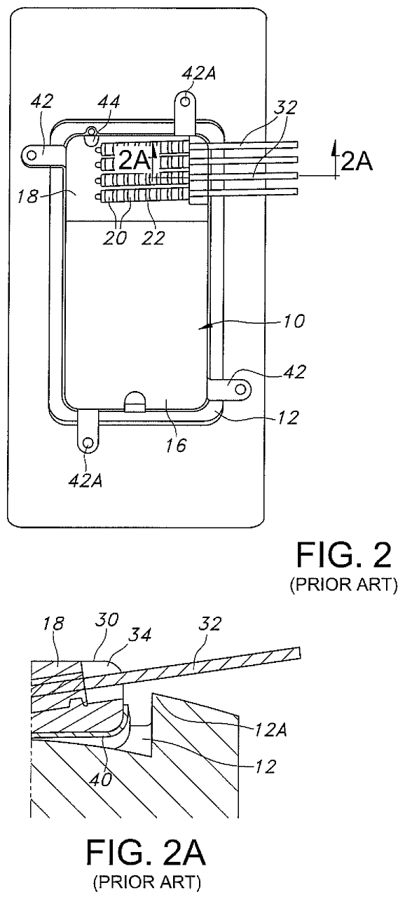

Turning now to the drawings, FIGS. 1, 2 and 2A show an active implantable medical device 10 that has been implanted in a cranial pocket 12 formed in the skull 14 of a patient. An exemplary operation is a craniotomy in which a bone flap is removed from the skull to access the brain of a patient to allow the surgeon to surgically implant the medical device 10, such as a deep brain neurostimulator for the treatment of Parkinson's disease, epilepsy, and cerebellar tremor, among other procedures.

The medical device 10 has a hermetically sealed housing 16 connected to a header assembly 18. The housing 16 provides an enclosure for the medical device including its control circuitry (not shown) and an electrical power source (not shown), such as a primary or rechargeable, secondary electrochemical cell connected to the control circuitry. Typically, the header assembly 18 is either molded directly onto a lid for the device housing 16 or it is a pre-molded assembly which is later secured to the housing lid. Suitable moldable polymeric materials for header body 110 include urethanes, such as e.g., TECOTHANE®, an aromatic polyether-based thermoplastic polyurethane sold by the Thermedics Polymeric Products Company of Wilmington, Mass.

In either case, the header assembly 18 serves as a structure that supports a plurality of co-axially aligned terminal blocks 20. An annular insulative member 22 between adjacent terminal blocks 20 prevents them from shorting to each other. The terminal blocks 20 are individually connected to the distal end of a feedthrough terminal pin 23 that extends into the device housing 16 where the pin is connected to the control circuitry. The arrangement shown in FIGS. 1 to 3, 5 and 7 to 9 has four rows of eight co-axially aligned terminal blocks 20 separated from each other by intermediate insulative members 22 and encased in the molded header assembly 18. However, those drawings are exemplary as the auxiliary strain relief members of the present invention can be used with medical devices that have more or less than four rows of aligned terminal blocks and with assemblies that have more or less than eight terminal blocks.

A set screw block 24 molded into the header assembly 18 is aligned with the terminal blocks 20 and the insulative members 22. The set screw block 24 extends from a proximal outwardly extending rim to a distal outwardly extending rim abutting the first terminal block 20. An intermediate, upstanding portion of the set screw block 24 has a threaded lateral opening 26 that is in open communication with a lumen 28 that extends to the proximal and distal rims.

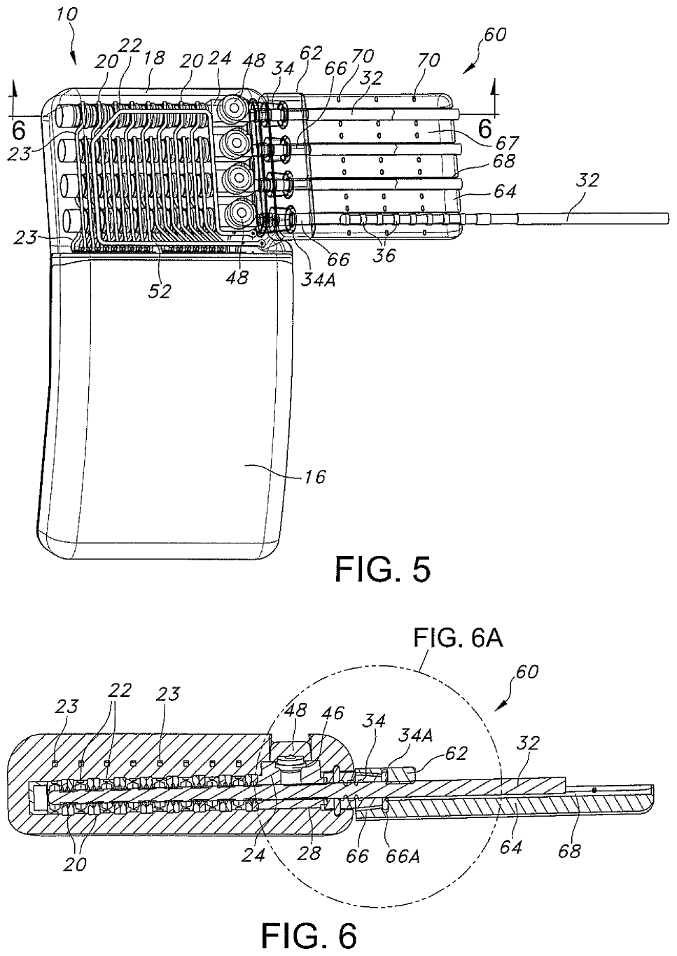

The header assembly 18 also has four proximally-facing openings 30 that align with the lumen 28 in a corresponding set screw block 24. This provides open communication for an implantable lead 32 to a respective row of the co-axially aligned terminal blocks 20. The drawings further show that the header assembly 18 supports a number of prior art or conventional cylindrically-shaped strain relief members 34 that are formed from a relatively pliable polymeric material, for example, polyurethane, and are received in one of the header openings 30, abutting the proximal end of a set screw block 24. The strain relief members 34 have an enlarged annular rim 34A with a diameter D₁ (FIG. 4) and extend distally from the header openings 30 a relatively short, but fixed distance. The strain relief members 34 help protect the implantable leads 32 from tension and stress.

In the exemplary embodiment shown in the drawings, each of the four implantable leads 32 has a number of electrical conductors (not shown) that extend along its length from at least one distal electrode (not shown) to a respective one of eight electrical contacts 36 located at a proximal end 38 of the lead. To connect an implantable lead 32 to the device's control circuitry, the proximal end 38 of the lead 32 is inserted into a strain relief member 34 and through the lumen 28 of a set screw block 24 to position its electrical contacts 36 inside a corresponding one of the aligned terminal blocks 20 encased in the header assembly 18. That way, the implantable lead 32 is detachably connected to the aligned terminal blocks 20 for electrically connecting the distal electrode to the device's control circuitry. When connected to a body organ or body tissue, the at least one distal electrode controlled by the control circuitry serves to deliver electrical stimulation to the body tissue of a patient or sense biological signals from the body tissue, or to both stimulate and sense.

As shown in FIGS. 1, 2 and 2A, the medical device 10 including its housing 16 and header assembly 18 connected to the four implantable leads 32 is nested in an open tray 40. The tray 40 has a number of outwardly extending tabs 42. With the open tray 40 holding the medical device 10, the tabs 42 are bent over and into contact with the skull 14. Each tab 42 is provided with an opening 42A that is configured to receive a threaded fastener (not shown) for securing the tray 40 to the skull 14. The tray 40 also has a pair of outwardly extending tabs 44 that are inwardly bendable into contact with the housing 16 for securing the medical device 10 in its nested position in the tray 40. These tabs 44 do not have an opening.

In a typical medical procedure, such as a craniotomy, the distance between the end of the fixed-length strain relief members 34 and the adjacent edge 12A of the cranial pocket 12 is relatively short. This means that the implantable leads 32 exit the strain relief members 34 and make a rather abrupt bend before resting against the edge 12A of the cranial pocket where they can rub against the skull 14. The concern is that this contact between the leads 32 and the edge 12A of the cranial pocket 12 could damage the polymeric outer insulation of the leads. If chafing and abrasion of the polymeric outer insulation of a lead is too severe, it could expose body fluids to the electrical conductors extending from the proximal electrical contacts 36 to the distal electrode. This could result in a short circuit condition for the implantable lead 32 which, of course, is undesirable.

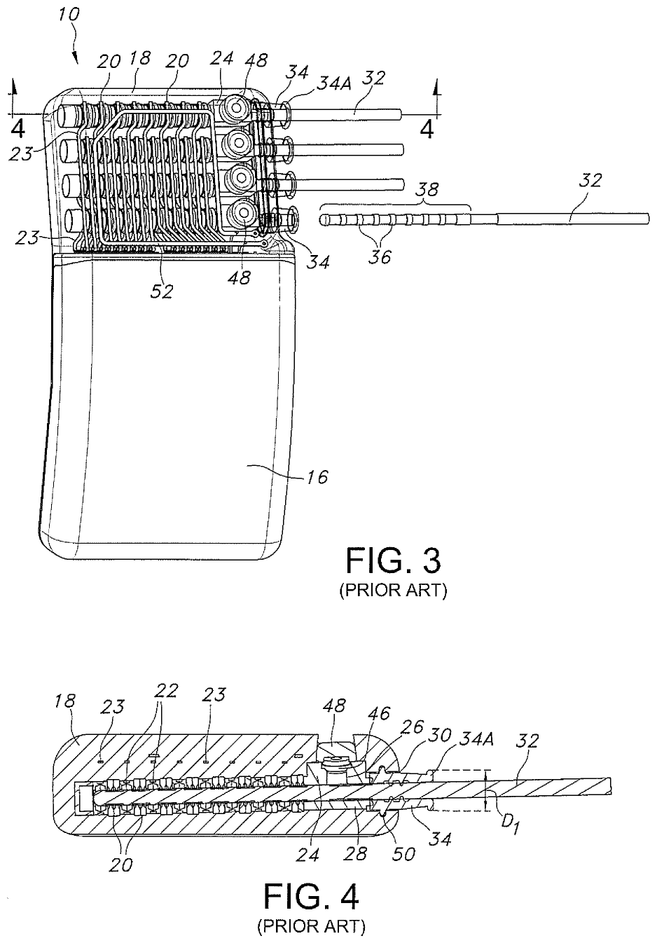

FIGS. 3 and 4 show an implantable lead 32 including its proximal electrical contacts 36 being moved into a conventional strain relief member 34 that is in open communication with the lumen 28 of a set screw block 24 and a row of aligned terminal blocks 20 and intermediate insulative members 22. FIG. 3 also shows three implantable leads 32 that have been inserted into the header assembly 18. FIG. 4 is an enlarged view that illustrates an implantable lead 32 that has already been inserted into a conventional strain relief 34 in open communication with the set screw block 24 which, in turn, is in open communication with eight co-axially aligned terminal blocks 20 and intermediate insulative members 22. With the implantable lead 20 received in the header assembly 18, a set screw 46 is threaded into the threaded lateral opening 26 in the upstanding portion of the set screw block 24 to prevent the lead from moving and losing electrical contact with the terminal blocks 20.

A polymeric cap 48 is seated in the threaded lateral opening 26, abutting the set screw 46. The polymeric cap 48 helps to keep body fluids from entering the lumen 28 of the set screw block 24 and then into the terminal blocks 20. A pair of side-by-side O-rings 50 molded into the header assembly 18 surrounds a stem portion of the strain relief member 24. These O-rings 50 help to keep body fluids from entering the set screw block 24 and then into the terminal blocks 20.

Finally, a loop coil or wire 52 connected to the control circuitry inside the device housing 16 serves as a bidirectional telemetry antenna for transmitting and receiving telemetry signals or as a charging coil for wirelessly charging the electrical power source for the implantable medical device 10. In another embodiment, the loop wire 52 is used to both charge the medical device and to conduct telecommunications with the medical device 10.

There are several drawbacks attributed to the use of the conventional strain relief members 34. For one, the portion of the strain relief member that extends outwardly beyond the header assembly 18 is relatively short. As shown in FIGS. 2 and 2A, this is the main reason that, after making an abrupt bend before resting against the edge 12A of the cranial pocket, the implantable lead 32 itself can rub against the skull 14.

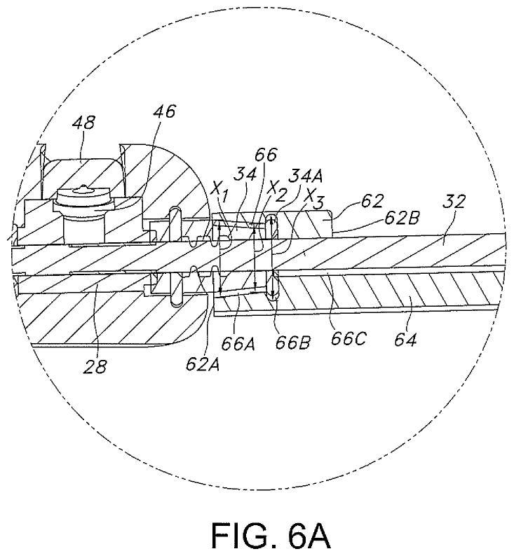

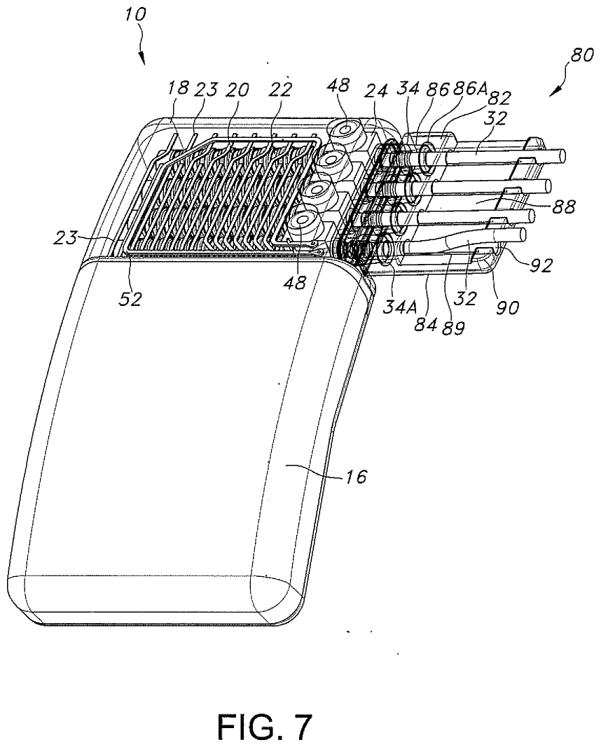

FIGS. 5, 6 and 6A illustrate a first embodiment of an auxiliary strain relief device 60 according to the present invention. The auxiliary strain relief device 60 is a unitary body made from a compliant polymeric material, for example, an MED 4750 implantable silicone commercially available from Nusil Technology, LLC, Carpinteria, California, and comprises a proximal manifold portion 62 connected to a distal platform portion 64. The manifold 62 has a number of shaped openings 66 that correspond to the number of conventional strain relief members 34 for the medical device 10. For example, if the medical device 10 has one conventional strain relief member 34, it is within the scope of the present invention that the manifold 62 of the auxiliary strain relief device 60 has one shaped opening 66. Accordingly, the medical device 10 can have fewer or more than the illustrated four conventional strain relief members 34 with the manifold 62 of the auxiliary strain relief device 60 having a like number of shaped openings 66.

Each shaped opening 66 in the manifold 62 extends along an opening axis from a proximal wall 62A to a distal wall 62B of the manifold and has an opening 66A with a diameter X₁ at the proximal wall 62A. The opening 66A leads to a tapered portion that extends downwardly towards and distally along the opening axis to a narrowed opening having a diameter X₂ immediately adjacent to an enlarged inner annular recess 66B having a diameter X₃. The diameter X₃ of the enlarged annular recess 66B perpendicular to the opening axis is greater than both the diameter X₁ of the opening 66A and the diameter X₂ of the narrowed opening X₂ immediately adjacent to the annular recess 66B. Further, the diameter X₁ of the opening 66A at the proximal wall 62A is either greater than, equal to or less than the diameter D₁ of the enlarged annular rim 34A. However, the compliant polymeric material comprising the auxiliary strain relief member 32 is compliant enough to permit the enlarged annular rim 34A to be moved into the opening 66A and slid down the tapered portion of the shaped opening until the enlarged annular rim 34A is snap-fit into the inner annular recess 66B. The annular recess 66B in turn is in open communication with a cylindrically-shaped open portion 66C that leads to the distal wall 62B of the manifold 62.

In use, with a manifold opening 66 in the auxiliary strain relief device 60 mated to a conventional strain relief member 34, the annular recess 66B receives the enlarged annular rim 34A of the strain relief member in a snap-fit connection. The snap-fit connection of each shaped opening 66 with a corresponding strain relief member 34 secures the auxiliary strain relief device to the plurality of conventional strain relief members connected to the header assembly 18 of the medical device 10.

The distal platform 64 of the auxiliary strain relief device 60 has a planar floor 67 interrupted by a number of spaced-apart open channels 68. Each channel 68 extends distally from the cylindrically-shaped open portion 66C of a shaped opening 66 in the manifold 62 to a distal edge of the platform. In that manner, the open channels 68 are aligned with a corresponding one of the manifold openings 66.

Then, with the number of shaped openings 66 in the auxiliary strain relief device 60 connected to the conventional strain relief members 34, an implantable lead 32 is connected to the device's header assembly 18 by moving its proximal portion 38 comprising the spaced-apart electrical contacts 36 into and through the cylindrically-shaped open portion 66C, then through the enlarged inner annular recess 66B and the tapered opening portion to the proximal wall 62A of the manifold portion 62 which, in turn, is in open communication with the conventional strain relief member 34 leading to the lumen 28 of the set screw block 24 and then the series of terminal blocks 20 and intermediate insulative members 22 of the header assembly.

With the proximal portion 38 of the implantable lead 32 securely connected to the header assembly 18, the distal portion of the lead 32 which extends distally from the manifold 62 is press-fit into an open channel 68 in the platform 64. In this position, the lower half of the lead 32 is nested in the open channel 68 with the upper half being exposed. However, as previously shown in FIG. 2A, it is only the lower half of the lead 32 that rubs against the edge 12A of the cranial pocket 12 in the skull 14.

An advantage of the auxiliary strain relief device 60 of the present invention is that the planar floor 67 helps to further reduce contact between the lead 32 and the auxiliary strain relief device 60 as the lead is inserted into an opening 66 in the manifold 62. The open space at the entrance to the manifold opening 66 helps to reduce the force needed to position the proximal portion 38 of the lead 32 and its electrical contacts 36 in the axially aligned terminal blocks 20.

FIG. 5 further illustrates that the distal platform 64 of the auxiliary strain relief device 60 has three spaced apart rows of perforations 70 that are aligned perpendicular to the open channels 68 in the platform. While three rows are shown, that is exemplary. The perforation rows 70 provide a convenient structure for trimming the platform 64 to a desired length. As shown in FIG. 2A, it is only desired to have the platform 64 extend a relatively short distance outwardly past the edge 12A of the cranial pocket 12. Excess length for the platform 64 is undesirable as it could interfere with the tray 40 holding the medical device 10 remaining in the desired position in the cranial pocket 12 after completion of the craniometry.

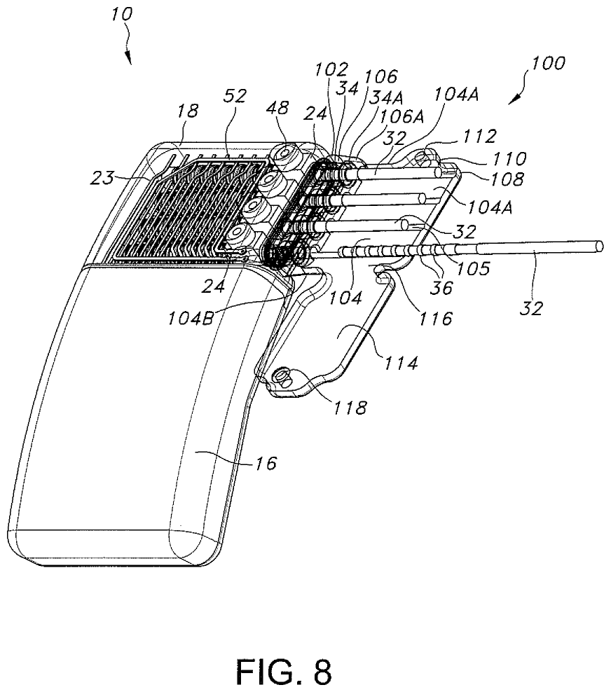

FIG. 7 illustrates another embodiment for an auxiliary strain relief device 80 according to the present invention. This auxiliary strain relief device 80 is similar to the auxiliary strain relief device 60 shown in FIGS. 5, 6 and 6A and is a unitary body made from a compliant polymeric material, for example, an MED 4750 implantable silicone. The auxiliary strain relief device 80 comprises a proximal manifold portion 82 connected to a distal platform portion 84. The manifold 82 is identical to the manifold 62 of the auxiliary strain relief device 60 with a number of shaped openings 86 that correspond to the number of conventional strain relief members 34 for the medical device 10. In the illustrated exemplary embodiment, there are four conventional strain relief members 34 so the manifold 82 has four openings 86 that are sized and shaped to snap-fit over and into a mated relationship with the strain relief members. In a similar manner as the shaped openings 66 in the auxiliary strain relief member 60, the manifold openings 86 each have an enlarged inner annular recess 86A intermediate a tapered open portion and a cylindrically-shaped open portion. The inner annular recess receives the enlarged annular rim 34A of a corresponding strain relief member 34 in a snap-fit connection that secures the auxiliary strain relief device 80 to the plurality of conventional strain relief members 34.

However, unlike the auxiliary strain relief device 60 shown in FIGS. 5, 6 and 6A, which has the open channels 68 extending distally from the manifold 62 to a distal edge of the platform 64, the platform 84 of the auxiliary strain relief device 80 shown in FIG. 7 has a planar floor 88 interrupted by a number of spaced-apart channels 89 that extend from the manifold to an upstanding wall 90 provided with a number of open grooves 92. The spaced apart channels 89 in the planar floor 88 are aligned with the grooves 92 in the upstanding wall 90.

In use, that portion of the lead 32 extending distally from the manifold 82 is received in an open channel 89 and then snap-fit into a groove 92 in the upstanding wall 90. That way, the planar floor 88 serves to further reduce the amount of contact between the lead 32 and the auxiliary strain relief device 80 as the lead is inserted into a shaped opening 86 in the manifold 82. The open space at the entrance to the shaped opening 86 helps to reduce the force needed to position the proximal portion 38 of the lead 32 and its electrical contacts 36 in the axially aligned terminal blocks 20.

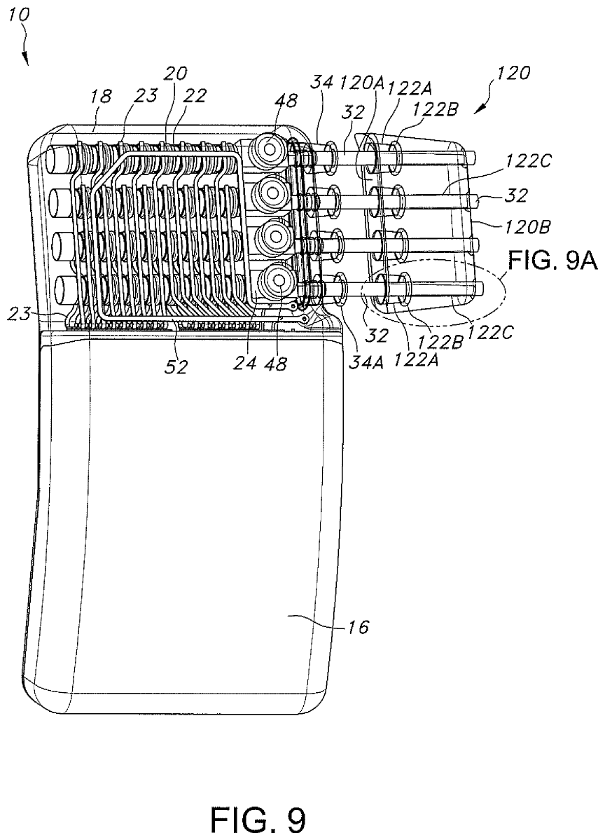

FIG. 8 illustrates another embodiment for an auxiliary strain relief device 100 according to the present invention. This auxiliary strain relief device 100 is a unitary body made from a compliant polymeric material, for example, an MED 4750 implantable silicone and, similar to the strain relief devices 60 and 80 shown in FIGS. 5, 6, 6A and 7, comprises a proximal manifold portion 102 connected to a distal platform portion 104. The manifold 102 is identical to the manifold 62 of the auxiliary strain relief devices 60 and 80. In a similar manner as the shaped openings 66 of the auxiliary strain relief devices 60 and 80, the shaped openings 106 each have an enlarged inner annular recess 106A intermediate a tapered opening portion and a cylindrically-shaped open portion. The inner annular recess receives the annular enlarged rim 34A of a corresponding strain relief member 34 in a snap-fit connection that secures the auxiliary strain relief device 100 to the plurality of conventional strain relief members 34.

Further, the platform 104 of the auxiliary strain relief device 100 shown in FIG. 8 has a planar floor 104A interrupted by a number of spaced-apart open channels 105 that extend from the manifold to a distal end of the planar floor. In use, the distal portion of the lead 32 extending from the manifold 102 is received in an open channel 105. That way, the planar floor 104A serves to further reduce the amount of contact between the lead 32 and the auxiliary strain relief device 100 as the lead is inserted into a shaped opening 106 in the manifold 102. The open space at the entrance to the shaped opening 106 helps to reduce the force needed to position the proximal portion 38 of the lead 32 and its electrical contacts 36 in the axially aligned terminal blocks 20.

This auxiliary strain relief device 100 further has an ear portion 110 extending outwardly from an upper edge 104A of the platform 104. The ear 110 has a blind bore 112. An outwardly extending moveable wing 114 is connected to a lower edge 104B of the platform 104 by a living hinge 116. The wing 114 supports an outwardly extending post 118. That way, after the leads 32 are secured in the header assembly 18 with the auxiliary strain relief device 100 snap-fit onto the conventional strain relief members 34 and with the leads nested in a respective open channel 108, the wing 114 is pivoted about its living hinge 116 until the post 118 is aligned with the blind bore 112 in the ear 110. The post 118 is then snap-fit into engagement with the blind bore 112 to secure the wing 114 over the leads 32 nested in the open channels 108. In that manner, the wing 114 serves as an additional structure that helps to secure the leads 32 extending through the conventional strain relief members 34 connected to the present invention strain relief device 100.

Additionally, it is within the scope of the present invention that the wing 114 connected to the blind bore 112 in the ear 110 of the auxiliary strain relief member shown in FIG. 8 can be adapted for use with the auxiliary strain relief devices 60 and 80 shown in FIGS. 5, 6, 6A and 7.

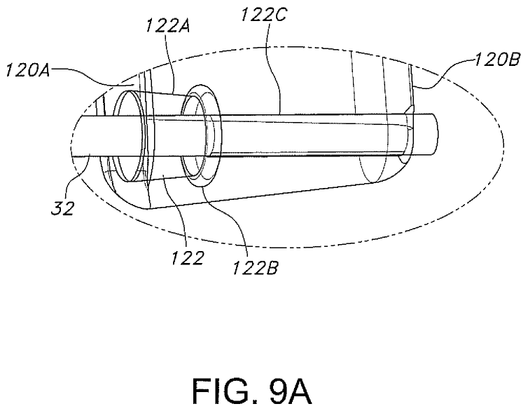

FIGS. 9 and 9A illustrate another embodiment for an auxiliary strain relief device 120 according to the present invention. This auxiliary strain relief device 120 is a unitary body made from a compliant polymeric material, for example, an MED 4750 implantable silicone. The auxiliary strain relief device 120 comprises a number of shaped proximal openings 122 leading to cylindrically-shaped distal openings 124 that correspond to the number of conventional strain relief members 34 for the medical device 10.

Each shaped opening 122 extends along an opening axis from a proximal wall 120A to a distal wall 120B of the auxiliary strain relief device 120 and has a tapered opening portion 122A that begins at the proximal wall 120A. The tapered opening portion 122A extends downwardly towards and distally along the opening axis to an enlarged inner annular recess 122B. The enlarged annular recess 122B has a diameter about the opening axis that is greater than the diameter of the tapered portion 122A immediately adjacent to the annular recess 122B. The annular recess 122B in turn is in open communication with a cylindrically-shaped open portion 122C that leads to the distal wall 120B.

In use, an implantable lead 32 is moved first through the cylindrically-shaped open portion 122C, the enlarged inner annular recess 122B and then the tapered opening portion 122A extending to the proximal wall 120A of the auxiliary strain relief device 120. However, the auxiliary strain relief device 120 is not yet connected to the strain relief member 34 of the medical device 10. The lead 32 in then moved into the header assembly 18 until the electrical contacts 36 at its proximal portion 38 are connected to the terminal blocks 20. With the lead 32 secured in this position after the set screw 46 is threaded into contact with the lead and the polymeric cap 48 is seated in the threaded lateral opening 26 against the screw, the auxiliary strain relief device 120 is moved in a proximal direction towards the medical device 10 until the annular recess 122B receives the enlarged annular rim 34A of the strain relief member in a snap-fit connection. The snap-fit connection of each shaped opening 122 in the auxiliary strain relief device 120 with a corresponding strain relief member 34 secures the auxiliary strain relief device to the plurality of conventional strain relief members connected to the header assembly 18 of the medical device 10. With the auxiliary strain relief device 120 connected to the conventional strain relief members 34, the resulting strain relief assembly provides the leads 32 with additional protection from tension and stress after the medical device 10 is implanted in a body and the distal electrodes are connected to body tissue.

Thus, various auxiliary strain relief members have been described. The auxiliary strain relief members are detachably connected to a conventional strain relief member to form a new strain relief assembly that improves protection of a lead for an extended length without unduly increasing the force needed to insert the lead into the header assembly. This is particularly important in medical devices that are implanted in the skull or near bones.

It is appreciated that various modifications to the inventive concepts described herein may be apparent to those skilled in the art without departing from the spirit and scope of the present invention as defined by the hereinafter appended claims.

What is claimed is:

1. An active implantable medical device (AIMD) assembly, the AIMD assembly comprising:
a) a medical device, comprising:
i) a device housing containing an electrical power source connected to control circuitry;
ii) a header assembly connected to the device housing, wherein the header assembly comprises a header supporting a plurality of co-axially aligned terminal blocks that are in open communication with a header opening leading into the header;
iii) a strain relief member connected to the header at the header opening, wherein the strain relief member has a strain relief opening that is in open communication with the header opening and the plurality of aligned terminal blocks;
b) an auxiliary strain relief device, comprising:
i) a manifold portion having a proximal wall spaced from a distal wall and at least one manifold opening that extends along an opening axis to the proximal and distal manifold walls, wherein the at least one manifold opening that is configured to detachably mate with the strain relief member of the medical device and comprises a tapered opening portion that begins at the proximal wall and extends downwardly towards and distally along the opening axis to an enlarged inner annular recess having a first diameter about the opening axis that is greater than a second diameter of the tapered portion immediately adjacent to the annular recess; and
ii) a platform portion that extends distally from the manifold portion, wherein the platform portion is provided with at least one open channel aligned with the at least one manifold opening.

2. The AIMD assembly of claim 1, wherein the tapered opening portion of the at least one manifold opening is in open communication with a cylindrically-shaped open portion of the manifold opening that leads to the distal wall of the manifold portion.

3. The AIMD assembly of claim 1, wherein the platform portion has a planar floor that is provided with the at least one open channel aligned with the at least one manifold opening.

4. The AIMD assembly of claim 3, wherein the at least one open channel extends to a distal end of the planar floor at a distal end of the platform portion.

5. The AIMD assembly of claim 3, wherein the at least one open channel extends along the planar floor to an upstanding wall at a distal end of the platform portion, and wherein the upstanding wall is provided with at least one open groove that is aligned with the at least one open channel.

6. The AIMD assembly of claim 1, wherein the platform portion is provided with at least one row of perforations that are aligned perpendicular to the at least one open channel of the platform portion.

7. The AIMD assembly of claim 6, wherein there are at least three spaced apart rows of perforations that are aligned perpendicular to the at least one open channel of the platform portion.

8. The AIMD assembly of claim 1, wherein an upper edge of the platform portion has an outwardly extending ear provided with a blind bore, and wherein an outwardly extending moveable wing is connected to a lower edge of the platform portion by a living hinge, the wing having a post that is received in the blind bore of the ear when the wing is pivoted about the living hinge to lay over the at least one open channel of the platform portion.

9. The AIMD assembly of claim 1, wherein the auxiliary strain relief device comprises a compliant implantable silicone.

10. An auxiliary strain relief device, comprising:
a) a manifold portion having a proximal wall spaced from a distal wall and at least one manifold opening that extends along an opening axis to the proximal and distal manifold walls, wherein the at least one manifold opening comprises a tapered opening portion that begins at the proximal wall and extends downwardly towards and distally along the opening axis to an enlarged inner annular recess having a first diameter about the opening axis that is greater than a second diameter of the tapered portion immediately adjacent to the annular recess; and
b) a platform portion that extends distally from the manifold portion, wherein the platform portion is provided with at least one open channel aligned with the at least one manifold opening.

11. The auxiliary strain relief device of claim 10, wherein the tapered opening portion of the at least one manifold opening is in open communication with a cylindrically-shaped open portion of the manifold opening that leads to the distal wall of the manifold portion.

12. The auxiliary strain relief device of claim 10, wherein the platform portion has a planar floor that is provided with the at least one open channel aligned with the at least one manifold opening.

13. The auxiliary strain relief device of claim 12, wherein the at least one open channel extends to a distal end of the planar floor at a distal end of the platform portion.

14. The auxiliary strain relief device of claim 12, wherein the at least one open channel extends along the planar floor to an upstanding wall at a distal end of the platform portion, and wherein the upstanding wall is provided with at least one open groove that is aligned with the at least one open channel.

15. The auxiliary strain relief device of claim 10, wherein the platform portion is provided with at least one row of perforations that are aligned perpendicular to the at least one open channel of the platform portion.

16. The auxiliary strain relief device of claim 10, wherein there are at least three spaced apart rows of perforations that are aligned perpendicular to the at least one open channel of the platform portion.

17. The auxiliary strain relief device of claim 10, wherein an upper edge of the platform portion has an outwardly extending ear provided with a blind bore, and wherein an outwardly extending moveable wing is connected to a lower edge of the platform portion by a living hinge, the wing having a post that is received in the blind bore of the ear when the wing is pivoted about the living hinge to lay over the at least one open channel of the platform portion.

18. The auxiliary strain relief device of claim 10, comprising a compliant implantable silicone.

19. An auxiliary strain relief device, comprising a manifold portion having a proximal wall spaced from a distal wall and at least one manifold opening that extends along an opening axis to the proximal and distal manifold walls, wherein the at least one manifold opening comprises a tapered opening portion that begins at the proximal wall and extends downwardly towards and distally along the opening axis to an enlarged inner annular recess having a first diameter about the opening axis that is greater than a second diameter of the tapered portion immediately adjacent to the annular recess.

20. The auxiliary strain relief device of claim 19, wherein the tapered opening portion of the at least one manifold opening is in open communication with a cylindrically-shaped open portion of the manifold opening that leads to the distal wall of the manifold portion.
